(19) United States
(12) Patent Application Publication
(10) Pub. No.: US 2010/0130579 A1
(43) Pub. Date: May 27, 2010

(54) CANCER THERAPY

(75) Inventors: Partha Banerjee, Rockville, MD (US); Shankar Jagadeesh, Gaithersburg, MD (US); Mikell Paige, Fairfax, VA (US); Kathryn Ditmer, Washington, DC (US); Milton L. Brown, Brookeville, MD (US)

Correspondence Address: WOLF GREENFIELD & SACKS, P.C., 600 ATLANTIC AVENUE, BOSTON, MA, 02210-2206, US

(73) Assignee: Georgetown University, Washington, DC (US)

(21) Appl. No.: 12/312,008

(22) PCT Filed: Oct. 23, 2007
(86) PCT No.: PCT/US2007/022441

§ 371 Date: Dec. 22, 2009

Related U.S. Application Data
(60) Provisional application No. 60/853,616, filed on Oct. 23, 2006.

(51) Int. Cl. A61K 31/403; C12N 15/01; A61P 35/00; C07D 491/052; C07D 209/82; A61K 31/407; C12Q 1/68

(52) U.S. Cl. 514/411; 435/441; 548/421; 548/440; 514/410; 435/6

(57) ABSTRACT

The present invention relates to methods of inducing expression of an epigenetically silenced gene, RASSF1A, in cells, particularly human cells, such as cancer cells. It also relates to methods of treating an individual, prophylactically or therapeutically, for cancer in which RASSF1A is epigenetically silenced.

### RELATED APPLICATIONS

This application claims the benefit under 35 U.S.C. §119(e) of U.S. Provisional Application Ser. No. 60/853,616, entitled “CANCER THERAPY,” filed Oct. 23, 2006, the entire teachings of which are incorporated herein by reference.

### BACKGROUND OF THE INVENTION

The incidence of prostate cancer has increased 142% in recent years. According to the American Cancer Society, approximately 180,000 men will be diagnosed with prostate cancer each year. (Landis, S H et al. CA Cancer J Clin (1999) 49: 8-31) Prostatic carcinoma is most invasive and the second leading cause of cancer death in men in USA. (Boring, C C et al. CA Cancer J Clin (1993) 43: 7-26) In the early stage of prostate cancer, the growth of prostatic carcinoma cells is androgen-dependent and can be effectively treated by hormone ablation either using surgical or pharmacological methods. (Huggins, C et al. Arch Surg (1941) 43: 209-223) However, hormone ablation therapy only causes a temporary regression of prostate tumors and invariably tumor become androgen-independent in 6-18 months. (Pfeifer G P et al. Biol Chem (2002) 383:907-14; Isaacs, J T Vitam Horm (1994) 49: 433-502) Therefore, androgen blockade is not the answer for treating prostate cancer. Additional approaches to treating prostate cancer are clearly needed.

### SUMMARY OF THE INVENTION

Described herein are compounds, such as mahanine or a derivative or equivalent thereof and carbazole compounds and derivatives thereof, whose structures are presented herein, which induce expression of RASSFIA and/or have anticancer effects. As described herein, Applicants have shown that mahanine induces expression of RASSF1A, an epigenetically silenced gene, in cancer cells. Applicants demonstrated that mahanine induces the expression of an epigenetically silenced gene, RASSF1A, in prostate cancer cells. They also have examined mahanine's effect on RASSF1A expression in skin, lung, pancreas, colon, breast and ovarian cancer cell lines. In all cases, mahanine induced epigenetically silenced gene RASSF1A. Applicants also demonstrate that RASSF1A regulates the transcriptional activity of a key cell cycle regulator, cyclin D1. This down-regulation of cyclin D1 is expected to be involved in cell cycle arrest of prostatic cancer cells at G0/G1. These results support the use of mahanine as a chemotherapeutic agent to prevent both androgen-sensitive and androgen-independent prostate cancer growth by inducing the expression of an epigenetically silenced gene, RASSF1A. The induction of RASSF1A expression by mahanine has significant biological consequences, particularly in cancer cells, where it can regulate transcriptional activation of a key cell cycle modulator, cyclin D1 and thereby control cell cycle progression, cell proliferation, and metastasis.

Also described herein are carbazole compounds (also referred to as synthetic carbazole compounds) useful in cancer therapy, such as the compounds whose structures are presented herein. These carbazole compounds can be synthesized using known methods.

The present invention relates to methods of inducing expression of an epigenetically silenced gene, RASSF1A, in cells, particularly human cells, such as cancer cells. It also relates to methods of treating an individual, prophylactically or therapeutically, for cancer in which RASSF1A is epigenetically silenced. In particular, it relates to treating individuals for prostate cancer, skin cancer, lung cancer, pancreatic cancer, colon cancer, breast cancer, ovarian cancer or other cancer in which RASSF1A is epigenetically silenced. In the method of the present invention, a drug or other agent that induces the expression of RASSF1A in cancer cells or precancer cells is administered in a therapeutically effective amount or dose to an individual at risk of developing cancer in which RASSF1A is epigenetically silenced or in whom cancer in which RASSF1A is epigenetically silenced has developed. The drug can be, for example, mahanine or a mahanine derivative or equivalent and the individual can be at risk for developing cancer (e.g., prostate cancer, skin cancer, lung cancer, pancreatic cancer, colon cancer, breast cancer, or ovarian cancer) in which RASSF1A is epigenetically silenced or an individual in whom such a cancer has developed.

Alternatively, the drug can be a carbazole compound described herein, such as compounds whose formula/structure are presented herein. A therapeutically effective amount or dose is one sufficient to reduce (partially or completely) the extent to which a cancer in which RASSF1A is epigenetically silenced occurs in an individual. For example, a therapeutically effective amount is one sufficient to prevent the occurrence of a cancer in which RASSF1A is epigenetically silenced in an individual at risk for developing such a cancer, limit the extent to which a cancer in which RASSF1A is epigenetically silenced occurs in an individual or reverse (partially or completely) a cancer in which RASSF1A is epigenetically silenced. A therapeutically effective amount is one that is sufficient to induce expression of RASSF1A to such an extent that it is not functionally “silent.” and, as a result, the cancer does not develop, develops to a lesser extent than would be the case in the absence of induction of RASSF1A expression or is reversed. In a particular embodiment, mahanine or a derivative or equivalent thereof is administered to a man who has prostate cancer in which RASSF1A is epigenetically silenced. Alternatively, a carbazole compound described herein, such as the compounds whose formula/structure are presented herein is administered to the man. In a further embodiment, mahanine or a derivative or equivalent thereof is administered to a man who is at risk of developing prostate cancer in which RASSF1A is epigenetically silenced, such as a man in whom PSA levels are elevated. Alternatively, a carbazole compound described herein, such as the compounds whose formula/structure are presented herein is administered to the man. The amount of mahanine or a derivative or equivalent thereof needed to produce the desired effect in a man will vary depending, for example, on his weight, age, general health status and the severity or stage of prostate cancer. The therapeutically effective amount can be determined empirically, using methods known to those of skill in the field. Mahanine or a derivative or equivalent thereof or a carbazole compound whose formula/structure is presented herein can be administered by a variety of routes, including parenteral (subcutaneous, intramuscular, intraorbital, intracapsular, intraspinal, intrasternal, intravenous) and nonparenteral routes, and can be given alone or in combination with other methods of treatment of prostate cancer (e.g., with other drugs, radiation, laser therapy). In all embodiments of the method, a combination of (a) mahanine, and/or (b) a mahanine derivative, analogue or equivalent of mahanine, and/or (c) one of more carbazole compounds whose formula/structure are presented herein can be administered.

In further embodiments of the invention, mahanine or a derivative or equivalent thereof or a carbazole compound whose structure/formula is presented herein can be administered to an individual at risk for other types of cancers in which RASSF1A is epigenetically silenced or in whom such cancer has occurred. Alternatively, a carbazole compound such as one or more of the carbazole compounds whose formula/structure are presented herein is administered. These include, but are not limited to, prostate cancer, skin cancer, lung cancer, pancreatic cancer, colon cancer, breast cancer, and ovarian cancer. The amount of mahanine or a derivative or equivalent thereof or of a carbazole compound whose structure/formula is presented herein needed to produce the desired effect in an individual will vary depending, for example, on his/her weight, age, general health status and the severity or stage of cancer. The therapeutically effective amount can be determined empirically, using methods known to those of skill in the field. Mahanine or a derivative or equivalent thereof or a carbazole compound whose structure/formula is presented herein can be administered by a variety of routes, including parenteral (subcutaneous, intramuscular, introrbital, intracapsular, intraspinal, intrasternal, intravenous) and nonparenteral routes, and can be given alone or in combination with other methods of treatment of prostate cancer (e.g., with other drugs, radiation, laser therapy).

A further embodiment of the invention is a method of identifying or screening for a drug that induces RASSF1A expression in cells, particularly cancer cells (e.g., prostate cancer cells, skin cancer cells, lung cancer cells, pancreatic cancer cells, colon cancer cells, breast cancer cells, and ovarian cancer cells) in which RASSF1A is epigenetically silenced. In the method of the present invention, a cancer cell in which RASSF1A is epigenetically silenced (referred to as a test cell), such as a prostate cancer cell, skin cancer cell, lung cancer cell, pancreatic cancer cell, colon cancer cell, breast cancer cell, or ovarian cancer cell in which RASSF1A is epigenetically silenced, is contacted with a candidate drug (a drug to be assessed for its ability to induce RASSF1A expression in a cancer cell), under conditions appropriate for cell growth or maintenance, and RASSF1A expression is determined. If RASSF1A expression is detected, the candidate drug is a drug that induces RASSF1A expression in the cells. RASSF1A expression in control cells, which are the same type of cancer cells as those contacted with the candidate drug and are maintained under the same conditions as the test cells except that they are not contacted with the candidate drug, can also be determined. Expression of RASSF1A in test cells and control cells is compared. Greater expression in test cells than in control cells indicates that the candidate drug is a drug that induces RASSF1A expression. A drug so identified can be further assessed for its activity (ability to induce RASSF1A expression) in vivo, such as by administering the drug to an appropriate animal model (e.g., a mouse or rat model of the cancer for which the drug is sought, referred to as the cancer of interest) and determining, using known methods, if RASSF1A expression occurs in cancer cells in the model. Additionally, the ability of the drug to reduce the occurrence of the cancer of interest (prevent its development in an individual at risk, reduce the extent to which it occurs and/or reverse the cancer once it has occurred) can be assessed in the model, using known methods.

In a specific embodiment, the present invention is a method of identifying a drug that induces expression of RASSF1A that has been epigenetically silenced in prostate cancer cells. The method comprises contacting a candidate drug with prostate cancer cells or a prostate cancer cell line (referred to as test prostate cancer cells), such as PC3 or LNCaP, under conditions appropriate for growth or maintenance of the cells and determining whether RASSF1A is expressed. If RASSF1A is expressed, the candidate drug is a drug that that induces RASSF1A expression in prostate cancer cells. RASSF1A expression in control prostate cancer cells, which are prostate cancer cells that are the same as those contacted with the candidate drug and are maintained under the same conditions as the test cells except that they are not contacted with the candidate drug, can also be determined. Greater expression in test cells than in control cells indicates that the candidate drug is a drug that induces RASSF1A expression. The ability of the candidate drug to repress transcriptional activity of cyclin D1 (a key cell cycle regulator) in prostate cancer cells can also be assessed as a way to identify a drug useful treating prostate cancer. A drug so identified can be further assessed for its activity (ability to induce RASSF1A expression and/or to repress transcriptional activity of cyclin D1) in vivo, such as by administering the drug to an appropriate animal model of prostate cancer (e.g., a mouse or rat model of prostate cancer) and determining, using known methods, if RASSF1A expression occurs in prostate cancer cells in the animal model. Additionally, the ability of the drug to reduce the occurrence of prostate cancer (prevent its development in an individual at risk, reduce the extent to which it occurs and/or reverse the cancer once it has occurred) can be assessed in the model, using known methods.

In specific embodiments, the invention relates to a method of treating an individual for cancer in which RASSF1A is epigenetically silenced, comprising administering to the individual a therapeutically effective amount of a drug that induces expression of RASSF1A in cancer cells or precancer cells in the individual, thereby limiting the extent to which a cancer in which RASSF1A is epigenetically silenced occurs in the individual or reversing (partially or completely) a cancer in which RASSF1A is epigenetically silenced in the individual. The cancer is, for example, prostate cancer, skin cancer, lung cancer, pancreatic cancer, colon cancer, breast cancer, ovarian cancer or other cancer in which RASSF1A is epigenetically silenced. The drug that is administered is wherein the drug is (a) mahanine, (b) a derivative equivalent or analogue of mahanine; (c) a compound of claim 1, 7, 8, or 9; or (d) a combination of two or more compounds of (a) (b) and/or (c). In another embodiment, the method is a method of treating prostate cancer in a man, comprising administering to the man a therapeutically effective amount of a drug (a) mahanine, (b) a derivative equivalent or analogue of mahanine; (c) a compound of claim 1, 7, 8, or 9; or (d) a combination of two or more compounds of (a) (b) and/or (c) whereby expression of epigenetically silenced RASSF1A is induced and prostate cancer occurs to a lesser extent than would be case in the absence of administration of mahanine of a derivative or analogue thereof.

In certain embodiments, a drug that is a derivative or analogue of mahanine, whose formula is shown below, is used. In alternative embodiments, the drug is a carbazole compound, such as a substituted carbazole, such as a compound whose formula/structure is presented herein. Such a drug can be administered to an individual in order to prevent or treat cancer, such as cancer in which RASSF1A is epigenetically silenced. The derivative or analogue is generally administered in a pharmaceutical composition, which can additionally comprise, for example, an appropriate carrier. Examples of derivatives or analogues of mahanine are described below.

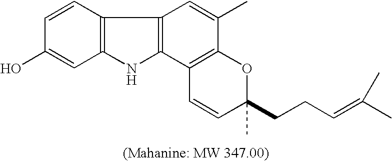

### BRIEF DESCRIPTION OF THE DRAWINGS

FIGS. 1A-1D. Mahanine induces RASSF1A in prostate and other cancer cells. FIG. 1A: Microarray analyses of PC3 cells treated with (2 mahanine (+) or vehicle (−) for 2 days, RNA was extracted and microarray analysis was performed. FIG. 1B: RT-PCR data showing the expression of RASSF1A and GAPDH in untreated normal prostate epithelial cells (PrEC) and human prostate cancer cells (PC3). FIG. 1C: PC3 (left panel) and LNCaP (right panel) cells were treated with 0, 1, 2 and 3 μg/ml mahanine for 3 days. RNA was extracted, and RT-PCR assays were performed to detect RASSF1A and GAPDH expression. Representative photograph from an experiment that was repeated thrice. Quantitative estimations of relative levels of RASSF1A mRNAs (lower panels) were determined by densitometric measurements of RT-PCR gels from three independent experiments after normalization with GAPDH. FIG. 1D: RT-PCR analyses of RASSF1A and GAPDH in A431, A549, ASPC-1, HT-29, MCF7 and SKOV-3 cells were performed after the treatment of 0, 2 and 3 μg/ml mahanine for 2 days. Columns, mean; bars, SEM. *, p<6.001, significantly different from control.

FIGS. 2A-2D. Mahanine down-regulates cyclin D1 expression in prostate and other cancer cells. FIG. 2A: Microarray analyses showing PC3 cells treated with (2 μg/ml) mahanine (+) or vehicle (−) for 2 days and RNA was extracted and microarray analysis was performed. FIG. 2B and FIG. 2C: PC3 (left panel) and LNCaP (right panel) cells were treated with 0, 1, 2 and 3 μg/ml mahanine for 3 days. RNA was extracted, and RT-PCR assays were performed to detect cyclin D1 and GAPDH mRNAs. Representative photograph from an experiment that was repeated thrice. Quantitative estimations of relative levels of cyclin D1 mRNAs (lower panel) were determined by densitometric measurements of RT-PCR gels from three independent experiments after normalization with GAPDH. FIG. 2D: RT-PCR analyses of cyclin D1 and GAPDH in A431, A549, ASPC-1, HT-29, MCF7 and SKOV-3 cells were performed after the treatment with 0, 2 and 3 μg/ml mahanine for 2 days. Columns, mean; bars, SEM. *, p<0.001, significantly different from control.

FIGS. 3A-3C. Mahanine down-regulates cyclin D1 protein in prostate cancer cells. FIG. 3A and FIG. 3B: Western blots showing cyclin D1 protein levels in PC3 (A) and LNCaP (B) cells treated with 0, 1, 2 and 3 μg/ml mahanine for 3 days. Protein lysates (50 μg) from PC3 and LNCaP cells were resolved on 12% SDS-PAGE, and immunoblots were probed with antibodies to cyclin D1. All immunoblots were re-probed with β-actin antibodies to ensure equal loading. Representative photographs from an experiment that was repeated thrice. Quantitative analyses of relative levels of cyclin D1 proteins are shown on the right panels. Columns, mean of three independent experiments; bars, SEM. *, p<0.01, significantly different from control. FIG. 3C: PC3 cells were plated on chamber slides and treated with or without 2 μg/ml mahanine for 2 days. Cells were then fixed in methanol, incubated with cyclin D1 antibody overnight, Alexi Fluor-conjugated secondary antibodies for 1 hour and counter stained with propidium iodide (PI). Slides were then mounted and examined using a fluorescence microscope. Photographs were taken at the same magnification (×20) and then transported to Photoshop. Representative photographs from an experiment that was repeated twice.

FIG. 4A-4B. Mahanine arrests prostate cancer cells at G0/G1 phase of cell cycle. To determine if down-regulation of cyclin D1 by mahanine treatments affect cell cycle, PC3 4(A) and LNCaP 4(B) cells were treated with 0, 1 and 2 μg/ml mahanine for 3 days. After treatment, flow cytometric analyses were performed. The percentage of cells in the G0/G1, S and G2/M-phase of the cell cycle were shown on right. FACS analysis of PC3 and LNCaP prostate cancer cells with vehicle or 2 μg/ml mahanine are shown in the left panel. Values are the mean from two independent experiments in duplicates.

FIG. 5. RASSF1A down-regulates cyclin D1 expression in prostate cancer cells but not other cyclins. PC3 cells were transiently transfected with 200 ng/ml empty vector (EV) or RASSF1A expression vector for 3 days. RNA was extracted, and RT-PCR assays were performed to detect RASSF1A, cyclin A1, B1, D1, E1 and GAPDH mRNAs. Representative photograph from an experiment that was repeated thrice. Quantitative estimations of relative levels of cyclin D1 and RASSF1A mRNAs were determined by densitometric measurements of RT-PCR gels from three independent experiments after normalization with GAPDH. Columns, mean; bars, SEM. *, p<0.001, significantly different from control.

FIGS. 6A-6B. Mahanine regulates the transcriptional activity of cyclin D1 and RASSF1A siRNA prevents mahanine-induced repression of cyclin D1 transcriptional activity. FIG. 6A: PC3 cells were transfected with 200 ng of full-length cyclin D1 promoter luciferase plasmids (−1745cyclin D1-Luc) or basic luciferase (PA3-Luc) plasmids and 10 ng of Renilla luciferase (pRL-TK-Luc) plasmids. Twenty-four hours after transfection cells were treated with 0, 1 and 2 μg/ml mahanine for 48 hours in normal growth media. FIG. 6B: PC3 cells were transfected with 200 ng of full-length cyclin D1 promoter luciferase plasmids or basic luciferase plasmids and 10 ng of Renilla luciferase (pRL-TK-Luc) plasmids with 200 ng of RASSF1A or RASSF1A siRNA with or without 2 μg/ml mahanine for 48 hours in normal growth media. After treatment, cells were harvested, and luciferase assays were performed. Relative cyclin D1 promoter activity was determined after normalization with Renilla luciferase activity. Luciferase activities in basic vector transfected cells were considered as 1.0. Columns, mean of three independent experiments with quadruplicate samples; bars, SEM. *, p<0.001, significantly different from control.

FIGS. 7A-7D. FIGS. 7A-7D show the dose-dependent inhibition of DNA synthesis with KED compounds. KED-3-63-1 and KED-3-81 have anticancer effects; KED-3-63-2 had no anti-cancer effects in the assay used.

FIG. 8. Shows three formulas of compounds that are the subject of this application.

### DETAILED DESCRIPTION OF THE INVENTION

Applicants demonstrated that mahanine, purified from Indian curry leaf, inhibits growth and induces apoptosis in both androgen-responsive, LNCaP and androgen-independent, PC3 prostate cancer cells in vitro. In addition, as described herein, they have shown that mahanine induces the expression of RASSF1A in human prostate cancer cells in a dose-dependent manner. The expression of RASSF1A is associated with a decrease in cyclin D1 message and protein levels and G0/G1 cell cycle arrest in prostate cancer cells. That is, there is an inverse relationship between RASSF1A and cyclin D1 expression. RASSF1A represses cyclin D1 transcription by inhibiting its promoter activity and addition of RASSF1A siRNA prevents this inhibition. Mahanine treatment also represses cyclin D1 transcriptional activity in prostate cancer cells. As described herein, mahanine induces the expression of an epigenetically silenced gene, RASSF1A, in prostate cancer cells. Expression of RASSF1A, in turn, is responsible for the repression/down-regulation of cyclin D1 expression and eventually the cell cycle arrest at the G0/G1 phase.

The etiology of human prostatic carcinoma remains largely undefined. However, it is becoming clear that epigenetic inactivation of various tumor suppressor genes could play a pivotal role in the development of various cancers, including prostate cancer. One such tumor suppressor is the Ras-association domain family 1 (RASSF1) gene. Two major isoforms of RASSF1, A and C, are produced from the human RASSF1 gene on chromosome 3p21.3 (1, 2). A diacylglycerol-binding domain is present at the amino-terminus of RASSF1A. The carboxy-terminus of RASSF1A contains a Ras-association domain. The biological function of RASSF1A is largely unknown. RASSF1C is a smaller protein (50 amino acids) that lacks the amino-terminal C1 domain. RASSF1C is thought to play a role in RAS-mediated cellular activities (3).

RASSF1A is probably the most frequently methylated gene described thus far in human cancers (4, 5). RASSF1A gene methylation has been reported in at least 37 tumor types. For example, methylation of RASSF1A is found in 80% of small cell lung cancers (2, 6), over 60% of breast tumors (2, 7, 8), 90% of liver cancers (9-11), 63% of pancreatic tumor (12), 40% of nonileal tumors (12), 69% of ileal tumors (12), 70% of primary nasopharyngeal cancers (13), 91% of primary renal cell carcinomas (14), 62% bladder tumor (15) and over 70% of prostate cancers (16-18).

Ectopic expression of RASSF1A in cancer cell lines that lack endogenous RASSF1A transcripts resulted in reduced growth of the cells in vitro and in nude mice, supporting a role for RASSF1A as a tumor suppressor gene (1, 2, 14, 16, 19-21). However, the mechanism underlying this tumor suppression is unclear. It has been demonstrated that the association of both RASSF1A and NORE1 (novel Ras effector 1) with the proapoptotic kinase, MST1 (mammalian sterile20-like 1) leads to apoptosis induction (22). Other studies have provided evidence that RASSF1A is a microtubule-binding protein that can stabilize microtubules and that its over-expression causes metaphase arrest by interacting with the components of the anaphase promoting complex (23-26).

RASSF1A KO-mice were viable and fertile but, as expected, were prone to spontaneous tumorigenesis (lymphoma, leukemia, lung adenoma, breast adenocarcinoma, rectal papiloma) in advanced age (18-20 months) (27). Shivakumar and associates have shown that the exogenous expression of RASSF1A induced cell cycle arrest in human lung cancer cells (H1299) at the G1 phase which was associated with the down regulation of cyclin D1 (28). RASSF1A also interacts with p120^{E4F}, a negative modulator of cyclin A expression (29).

Another study demonstrated that RASSF1A suppress the c-Jun-NH2-kinase pathway to inhibit cell cycle progression (30). These findings support a role for RASSF1A in the regulation of cell cycle. The restoration of RASSF1A expression in tumor cell lines impairs their tumorigenicity (14, 16) and, therefore, factors that restore RASSF1A expression have immense potential in preventing tumor growth and, thus, in cancer prevention and therapy.

Murraya koenigii, a small shrub, is widely found in East Asia. It is popularly known in India as “curry leaf plant” and the leaves are heavily consumed in both raw and cooked forms. In a recent study, Applicants evaluated the anti-proliferative activity of mahanine, isolated and purified from M. koenigii, in human prostate cancer cells. They demonstrated that mahanine inhibits growth in a dose-dependent manner and at a greater concentration (3 μg/ml), induces apoptosis in both androgen-responsive, LNCaP and androgen-independent, PC3 cells (33).

As described herein, mahanine induces the expression of an epigenetically silenced tumor suppressor gene, RASSF1A, in human prostate cancer cells and down-regulates cyclin D1 to arrest the cells at the G0/G1 phase of the cell cycle.

Applicants have shown that mahanine induces an epigenetically silenced gene, RASSF1A, in prostate cancer cells and induction is, in turn, associated with cell cycle arrest. In addition, Applicant showed that RASSF1A acts as a transcriptional inhibitor of a key cell cycle regulator, cyclin D1.

Using two human prostate cancer cell lines, PC3 and LNCaP, they demonstrated that mahanine decreased cyclin D1 message and protein levels in a dose-dependent manner and eventually arrested the cells at the G0/G1 phase of the cell cycle. The down-regulation of cyclin D1 expression and transcriptional activity in prostate cancer cells is consistent with previous reports that have shown that exogenous RASSF1A induces a G1 arrest.

Applicants show here that RASSF1A inhibits cyclin D1 protein accumulation by down-regulating cyclin D1 transcriptional activity. Therefore, by inducing the epigenetically silenced gene, RASSF1A, mahanine regulates cyclin D1, a key cell cycle regulator and arrests cell at G0/G1 phase.

Questions might arise about how RASSF1A regulates cyclin D1 transcription. Transcription of the cyclin D1 gene is induced via distinct DNA sequences in its promoter by diverse mitogenic and oncogenic signaling pathways including Ras, Src, Stat3, Stat5 and Erbb2 (37). Several transcription factors, such as CREB, AP-1, β-catenin/Tcf-1, have been shown to interact with the cyclin D1 promoter (35, 38). It is possible that one or more of these signaling pathways and transcription factors are involved in the transcriptional regulation of cyclin D1 by RASSF1A. The activation of cyclin D1 gene transcription is dependent on the activation of Ras, Raf, mitogen activated protein kinase-kinases (MEK1 and MEK2), Akt and the sustained activation of extracellular signal regulates protein kinases (ERKs) (37). On the other hand, cyclin D1 degradation is mediated by phosphorylation-triggered ubiquitin-dependent proteolysis (39). Glycogen synthase kinase 3β (GSK-3β) catalyzes the phosphorylation of cyclin D1 on Thr286 and redirects the protein from the nucleus to the cytoplasm (39).

Applicants have previously demonstrated (33) that mahanine deactivated Akt in prostate cancer cells. Activated Akt deactivates GSK3β by phosphorylation. Therefore, it is possible that in addition to the transcriptional repression of cyclin D1 by RASSF1A, mahanine also deactivates Akt, which would eventually activate GSK-3β to degrade cyclin D1.

Overexpression of cyclin D1 is a common event in various forms of cancer, including prostate cancer (40-42). The overexpression of cyclin D1 leads to enhanced organ growth in mice (43). Transient transfection of hepatocytes with cyclin D1 leads to vigorous proliferation and more than 50% increase in liver mass within 6 days (44). Conversely, cyclin D1 knockout mice are smaller than wild-type mice and mice with the homozygous deletion of the p27 gene (which inhibits cyclin D1/Cdk4/6 complexes) show gigantism and enhanced organ size (45). Moreover, the expression of cyclin D1 modulates invasive ability by increasing matrix metalloproteinase (MMP-2 and MMP-9) activity and motility in glioma cells (46). Furthermore, some studies have shown that over-expression of cyclin D1 is associated with metastatic prostate cancer to bone (47). These finding suggest that in addition to its well defined role in cell cycle progression, cyclin D1 may also play a role in the regulation of cell growth and metastasis. Therefore, the repression of cyclin D1 transcription by mahanine via RASSF1A would allow mahanine to modulate prostate cancer cell proliferation and/or its invasive potential.

Although RASSF1A is epigenetically silenced in many carcinomas, and its silencing is believed to be associated with carcinogenesis, the mechanism of RASSF1A silencing is largely unknown. It has been demonstrated that promoter hypermethylation is the major cause of RASSF1A gene silencing in variety of human cancers (2, 4-18). Since DNA methyltransferases (DNMTs) methylate the DNA, and mahanine induces the expression of RASSF1A, it is tempting to speculate that mahanine may inhibit DNMTs to prevent DNA methylation and induces the expression of RASSF1A. Further investigation of the effect of mahanine on DNMTs (e.g., whether it inhibits the expression and/or activity of DNMTs) would be very relevant. At higher concentrations (3 μg/ml), mahanine induces cell death in prostate cancer cells by the activation of caspase-3. Induction of RASSF1A is also greater at this concentration of mahanine in prostate cancer and various non-prostatic cancer cells that Applicants have evaluated. Now there is evidence that both NORE1 and RASSF1A associate with the proapoptotic kinase, Mst1 and this interaction is involved in the apoptotic process (48). Since Mst1 is both a caspase-3 cleavage target and an enhancer of caspase-3 activation (49), it is possible that in addition to regulating the cell cycle, mahanine can induce RASSF1A to induce cell death. Applicants have previously shown that mahanine greatly increased caspase-3 cleavage and activation in PC3 cells.

Carbazole compounds of the invention include compounds having the following structure (I):

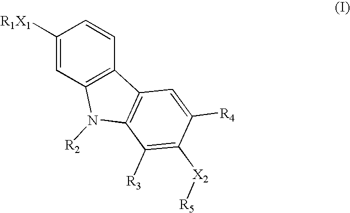

including stereoisomers and pharmaceutically acceptable salts thereof; wherein
R₁, R₂, R₃ and R₄ can be the same or different and each is H, alkyl, alkenyl, CF₃, cycloalkyl, or benzyl, optionally substituted; R₅ is alkyl, alkenyl, or benzyl, optionally substituted; or, R₄ and R₅ are joined together to form a six-membered ring, optionally substituted; X₁ and X₂ can be the same or different and each is O, C(R₆)(R₇), NR₈, S, or C═O; R₆ and R₇ can be the same or different and each is absent, H, alkyl, or alkenyl; and R₈ is absent, H, alkyl, C═O, or S═O. (O, Oxygen; C, carbon; N, nitrogen; S, sulfur)

In some embodiments, X₁ and X₂ are 0. In some embodiments R₂ is H. In some embodiments R₁ is CH₃ and R₅ is benzyl. In some embodiments R₃ is H or CH₃ and R₄ is H or CH₃. In some embodiments the compound comprises at least one benzyl group, wherein the benzyl group comprises a phenyl ring. In some cases, the phenyl ring may be optionally substituted with at least one OH, NH₂, NHR₉, OCH₃, or alkyl group, wherein R₉ is H, alkyl, C═O, or S═O.

In some embodiments, the compound has the following structure (II):

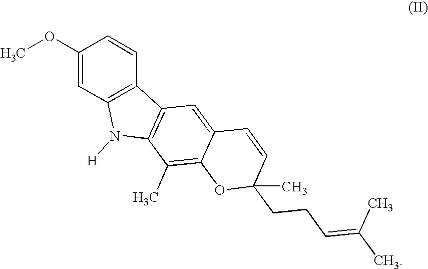

In some embodiments, the compound has the following structure (III):

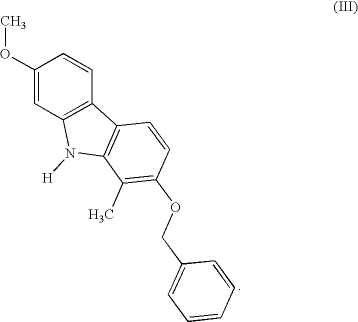

In some embodiments, the compound has the following structure (IV):

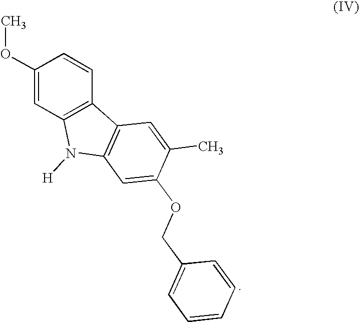

As used herein, the term “alkyl” refers to an aliphatic hydrocarbon group which may be straight, branched, or cyclic (e.g., “cycloalkyl”), having from 1 to about 10 carbon atoms in the chain, and all combinations and subcombinations of ranges therein. The term “alkyl” includes both “unsubstituted alkyls” and “substituted alkyls,” the latter of which refers to alkyl moieties having substituents replacing a hydrogen on one or more carbons of the backbone. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclohexyl, and the like.

The term “methyl” refers to the group “—CH₃.”

The term “alkenyl” refers to unsaturated aliphatic groups analogous in length and possible substitution to the alkyls described above, but that contain at least one double bond.

In some embodiments, the compounds described herein may be “optionally substituted,” that is, the compounds may be substituted or unsubstituted.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term stereochemically isomeric forms of compounds, as used herein; include all possible compounds made up of the same atoms bonded by the same sequence of bonds but having different three-dimensional structures which are not interchangeable, which the compounds may possess. Unless otherwise mentioned or indicated, the chemical designation of a compound encompasses the mixture of all possible stereochemically isomeric forms that the compound can take. The mixture can contain all diastereomers and/or enantiomers of the basic molecular structure of the compound. All stereochemically isomeric forms of the compounds both in pure form or in admixture with each other are intended to be embraced within the scope of the present invention.

Some of the compounds may also exist in their tautomeric forms. Such forms although not explicitly indicated in the above formula are intended to be included within the scope of the present invention.

The compounds (e.g., mahanine, a derivative or equivalent of mahanine, a carbazole compound whose structure/formula is presented herein or a combination of two or more of the foregoing) for example, one or more compounds represented by formula I, II, III or IV can be administered (alone or in combination with mahanine a derivative or equivalent thereof) are administered in effective amounts. An effective amount is a dosage of the compound(s) or therapeutic agent(s) sufficient to provide a medically desirable result. An effective amount means that amount necessary to delay the onset of, inhibit the progression of or halt altogether the onset or progression of the particular condition or disease being treated. In the treatment of cancer, for example, in general, an effective amount will be, for example, that amount necessary to inhibit cancer cell replication, reduce cancer cell load, or reduce one or more signs or symptoms of the cancer. When administered to a subject, effective amounts will depend, of course, on the particular cancer being treated; the severity of the cancer; individual patient parameters including age, physical condition, size and weight, concurrent treatment, frequency of treatment, and the mode of administration. These factors are well known to those of ordinary skill in the art and can be addressed with no more than routine experimentation. In some embodiments, it is preferred to use the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

Also the subject of this invention are compositions, such as pharmaceutical compositions or formulations which comprise (1) at least one of the following: mahanine; a derivative or equivalent of mahanine; a carbazole compound whose structure/formula is presented herein and (2) an appropriate (pharmaceutically useful) carrier. Actual dosage levels of active ingredients in the pharmaceutical compositions of the invention can be varied to obtain an amount of the active compound(s) that is effective to achieve the desired therapeutic response for a particular patient, compositions, and mode of administration. The selected dosage level depends upon the activity of the particular compound, the route of administration, the severity of the condition being treated, the condition, and prior medical history of the patient being treated. However, it is within the skill of the art to start doses of the compound at levels lower than required to achieve the desired therapeutic effort and to gradually increase the dosage until the desired effect is achieved.

The compounds and pharmaceutical compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea.

Compounds of the present invention also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of a compound of this invention include powders, sprays, ointments, and inhalants as described herein. The active compound is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from subcutaneous or intramuscular injection. This result can be accomplished by the use of a liquid suspension of crystalline or amorphous materials with poor water solubility. The rate of absorption of the drug then depends upon its rate of dissolution which, in turn, may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

In such solid dosage forms, the active compound is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In a most preferred embodiment, the excipient or carrier increases uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The active compounds also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the active compounds, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the compounds of the invention. The compound is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl. 5): s. 143-146 (1989)(endothelin-1); Hubbard et al., Annals of Internal Medicine 3:206-212 (1989)(α1-antitrypsin); Smith et al., J. Clin. Invest. 84:1145-1146 (1989) (al proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated.

Formulations suitable for use with a nebulizer, either jet or ultrasonic, typically comprise a compound of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the inhibitor compound suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the inhibitor and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the compounds and composition of the invention also is contemplated. Nasal delivery allows the passage of the compound or composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the compounds of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of compounds across cell and/or nuclear membranes, compositions of relatively high hybrophobicity are preferred. Compounds can be modified in a manner which increases hydrophobicity, or the compounds can be encapsulated in hydrophobic carriers or solutions which result in increased hydrophobicity.

The invention is exemplified by the following Example.

### EXAMPLE

### Materials and Methods

The following methods and materials were used in work described herein. Cell line and cell growth assay.

PC3, LNCaP, A431, A549, ASPC-1, HT-29, MCF7 and SKOV-3 cells (ATCC, Manassas, Va.) were grown in IMEM without phenol red (Biofluids, Rockville, Md.) supplemented with 10% fetal bovine serum (Quality Biologicals, Gaithersburg, Md.), 2 mM glutamine, 100 units/ml penicillin G sodium and 100 μg/ml streptomycin sulfate (Sigma, St. Louis, Mo.) in the presence of 5% CO₂ at 37° C.

Microarray analysis: Total RNA was isolated from DU145 cells treated with or without mahanine using TRIZOL reagent (Invitrogen Corp., Carlsbad, Calif.). Total RNA was purified using Qiagen RNeasy mini columns. RNA was evaluated by electrophoresis before continuing with probe synthesis and hybridization. Total RNA (3 μg) was reverse transcribed, and double-stranded cDNA probes were generated by biotin-16-dUTP incorporation using the TrueLabeling-AMP2.0 kit (SuperArray, Frederick, Md.), according to the manufacturer's instructions. The cDNA probes were denatured at 95° C. for 2 minutes. Jak/STAT Signaling Pathway Microarray membranes (SuperArray) were prehybridized in GEAprehyb (SuperArray) with heat-denatured sheared salmon sperm for 2 h at 60° C. Labeled cDNA probes were hybridized overnight at 60° C. with continuous agitation. Following repetitive washing in saline-sodium citrate/SDS, hybridized cDNA probes were detected by chemiluminescence. Membranes were blocked for nonspecific binding with GEAblocking solution Q (SuperArray). Bound biotinylated cDNA probe was detected with alkaline phosphatase-conjugated streptavidin and CDP-Star chemiluminescent substrate (SuperArray). Images of the membranes were captured using a Fuji LAS-1000 Imager (Tokyo, Japan). Data were further processed with GEArray Analyzer software (http://www.superarray.com), correcting for background noise by subtraction of the minimum value and normalizing to the maximum value of each individual array. Genes were considered present if the expression level was greater than two times that of the blank negative control. Genes were considered to be differentially expressed in control and mahanine treated cells if the change was greater than 2.0-fold and two of the three samples followed the down-regulation or up-regulation.

Western blots analysis: Protein lysates were prepared from PC3 and LNCaP cells that were treated with or without mahanine. The lysates were resolved on 12% SDS-PAGE and transferred to nitrocellulose membranes. The membranes were probed with 1:1000 dilution of cyclin D1 (Santa Cruz Biotechnology, Santa Cruz, Calif.) overnight at 4° C. Each blot was re-probed with 1:10,000 dilution of β-actin (Sigma Chemicals, St. Louis, Mo.). Images of the membranes were captured using a Fuji LAS-1000 Imager (Tokyo, Japan) and imported into Adobe Photoshop. Band intensities were quantified by utilizing ImageJ software (NIH, Bethesda, Md.).

Immunofluorescence staining: PC3 cells were plated onto chamber slides and treated with vehicle or mahanine (2 μg/ml) for 2 days. The cells were fixed in methanol, air-dried, and re-hydrated with PBS. 0.2% BSA was used for blocking and then the cells were incubated with the primary antibody (cyclin D1; 1:200 dilutions) overnight at 4° C. The cells were washed three times with PBS and incubated with 4 μg/ml Alexa Fluor 488 labeled donkey anti-rabbit IgG (Molecular probes/Invitrogen, Carlsbad, Calif.), for one hour. The cells were washed again three times with PBS and subsequently counterstained with 0.5 μg/ml propidium iodide (PI), viewed and photographed using a camera-equipped fluorescent microscope (ZEISS AxioPlan2 Imaging System, Jena, Germany) and images were transferred to photoshop.

Reverse transcriptase polymerase chain reaction (RT-PCR): RNA was extracted from PC3, LNCaP, A431, A549, ASPC-1, HT-29, MCF7 and SKOV-3 cells with TRIzol solution as suggested by the manufacturer (Invitrogen, Carlsbad, Calif.). Genes of interest were amplified using 500 ng of total RNA reverse-transcribed to cDNA using a Superscript II kit (Invitrogen) with random hexamers. Human-specific primers were designed using the Primer Quest program and purchased from Integrated DNA Technologies, Inc (Coralville, Iowa). Their sequences and product band sizes are: cyclin D1 forward primer 5′-CACACGGACTACAGGGGAGT-3′ (SEQ ID NO.: 1); cyclin D1 reverse primer 5′-AGGAAGCGGTCCAGGTAGTT-3′ (475 bp) (SEQ ID NO.: 2); cyclin A1 forward primer 5′-AAGG AGTGTGCGTCAGGACT-3′ (SEQ ID NO.: 3); cyclin A1 reverse primer 5′-CAACGTGCAGAAGCCT AT GA-3′ (413 bp) (SEQ ID NO.: 4); cyclin B1 forward primer 5′-CGGGAAGTCACTGGAAACAT-3′ (SEQ ID NO.: 5); cyclin B1 reverse primer 5′-CCGACCCAGACCAAAGTTTA-3′ (315 bp) (SEQ ID NO.: 6); and cyclin E1 forward primer 5′-AAGTGGATGGTTCCATTTGC-3′ (SEQ ID NO.: 7); cyclin E1 reverse primer 5′-TTTGATGCCATCCACAGAAA-3′ (399 bp) (SEQ ID NO.: 8); and GAPDH forward primer: 5′-CCA CCCATGGCAAATTCCATGGCA-3′ (SEQ ID NO.: 9); GAPDH reverse primer: 5′-TCTAGACGGCAG GTCAGGTCCACC-3′ (598 bp) (SEQ ID NO.: 10). PCRs were initiated at 94° C. for 2 min, followed by 28 cycles of 94° C. for 1 min, 1 min annealing temperature, 72° C. for 1 min, and final extension at 72° C. for 5 min. The annealing temperature for cyclin B1 and cyclin E1 was 55° C., and 60° C. for cyclin D1, cyclin A1 and GAPDH. Primers and PCR conditions for RASSF1A and 1C are used as described by Lee and associates (34). After amplification, PCR products were separated on 1.5% agarose gels and visualized by ethidium bromide fluorescence using the Fuji LAS-1000 Imager. Images were captured and imported to Adobe Photoshop. Band intensities were quantified by using ImageJ software (NIH, Bethesda, Md.).

Cyclin D1 promoter activity assay: PC3 cells were transfected with 200 ng of cyclin D1 promoter-luciferase (−1745-cyclin D1-Luc) (35) construct using GeneJammer transfection reagent (Stratagene, La Jolla, Calif.). PC3 cells were also co-transfected with 200 ng of RASSF1A (26, 27) or RASSF1A siRNA plasmids (36). After 48 hours, luciferase activity was measured in cell lysates in a microplate luminometer using the Dual Luciferase Assay kit (Promega, Madison, Wis.) according to the manufacturer's protocol. Luciferase activity was normalized to Renilla luciferase activity by co-transfection of pRL-TK plasmid (10 ng).

Statistical analyses: All data was derived from at least three independent experiments and statistical analyses were conducted using Prism 3 GraphPad software. Values were presented as mean±SEM. Significance level was calculated using the one-way Analysis of Variance (ANOVA) followed by the Dunnett post-test with an assigned confidence interval of 95%. P-value<0.05 was considered significant.

Sources of materials: Applicants acknowledge each of the following: Dr. Richard Pestell (Kimmel Cancer Center, Thomas Jefferson University, Philadelphia, Pa.) for his generous gift of the full-length cyclin D1 promoter luciferase construct and Dr. Ying Huang (State University of New York, Syracuse, N.Y.), Dr. Gerd P. Pfeifer (Beckman Research Institute, City of Hope National Medical Center, Duarte, Calif.) and Dr. Dae-Sik Lim (Korea Advanced Institute of Science and Technology, Daejeon, Korea) for providing the EGFP-tagged-RASSF1A, EGFP-empty vector and RASSF1A siRNA expression vector, respectively.

### Example 1

### Mahanine Induces an Epigenetically Silenced Gene RASSF1A in Prostate Cancer and Various Non-Prostatic Cancer Cells

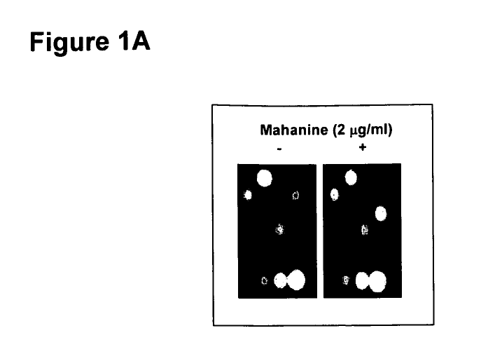

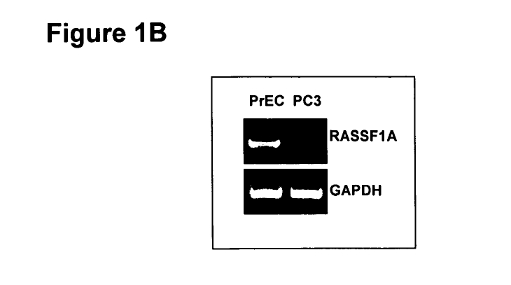

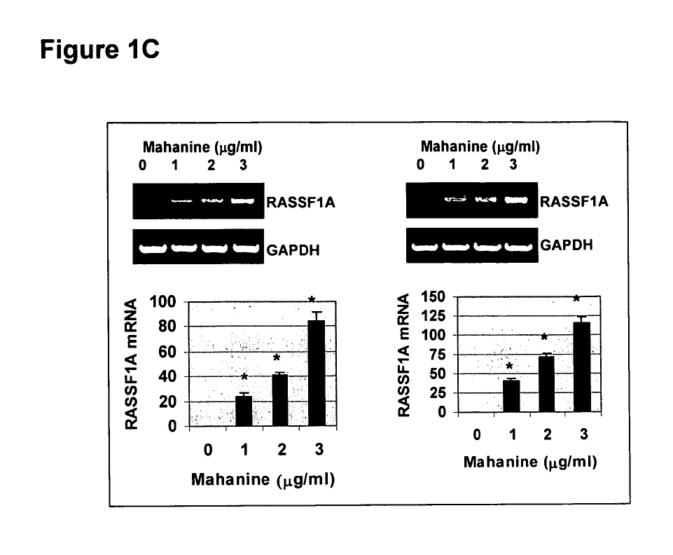

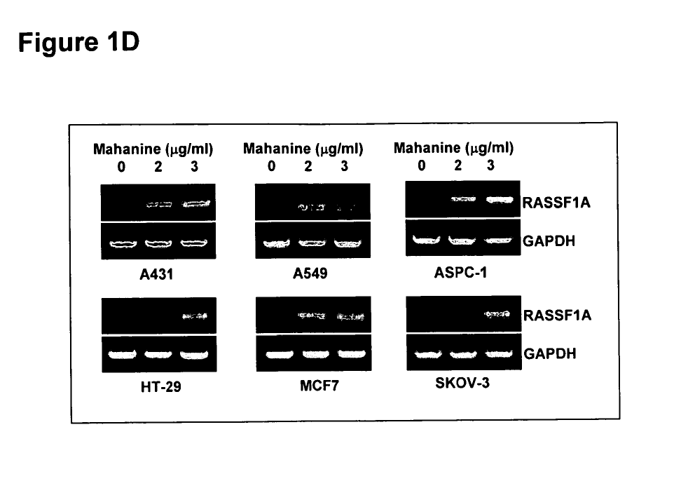

Applicants performed a gene array analysis by using 2 μg/ml mahanine-treated human prostate cancer cells, PC3. Results showed that the RASSF1 gene was dramatically induced in mahanine-treated PC3 cells compared to the vehicle-treated control PC3 cells (FIG. 1A). RASSF1A is an epigenetically silenced gene and, therefore, Applicants also examined its expression in normal prostate epithelial cells (PrEC). Using equal amounts of RNA from PrEC and PC3 cells, Applicants observed that RASSF1A is highly expressed in normal prostate epithelial cells (PrEC), but not in prostate cancer (PC3) cells (FIG. 1B). Re-examination of microarray analysis by RT-PCR demonstrated that mahanine induces the expression of the RASSF1A gene in PC3 cells in a dose-dependent manner (20-80 folds) (FIG. 1C). This event is not cell type specific; Applicants also observed a similar effect of mahanine in another human prostate cancer cell line, LNCaP (FIG. 1C). Since RASSF1A gene silencing occurs in 37 different cancer types, Applicants were interested in evaluating the effects of mahanine in various non-prostatic human cancer cell lines. Similar to prostate cancer cells, RASSF1A was absent in epidermoid (A431), lung (A549), pancreatic (ASPC-1), colon (HT-29), breast (MCF7) and ovarian (SKOV-3) cells and mahanine treatment for 2 days induced RASSF1A expression in all of these cancer cells (FIG. 1D). These results suggest that mahanine has the ability to induce the epigenetically silenced gene RASSF1A, not only in prostate cancer cells, but also in various non-prostatic cancer cells.

### Example 2

### Mahanine Inhibits the Expression of Cyclin D1, but not Other Cyclins, in Human Prostate Cancer Cells

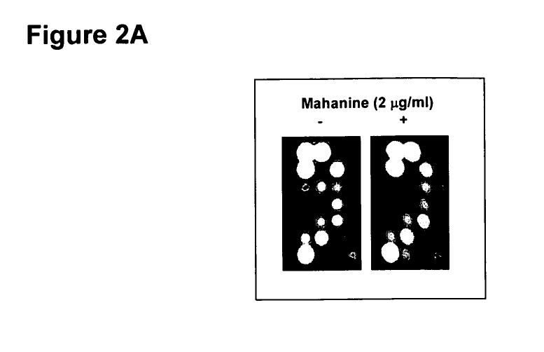

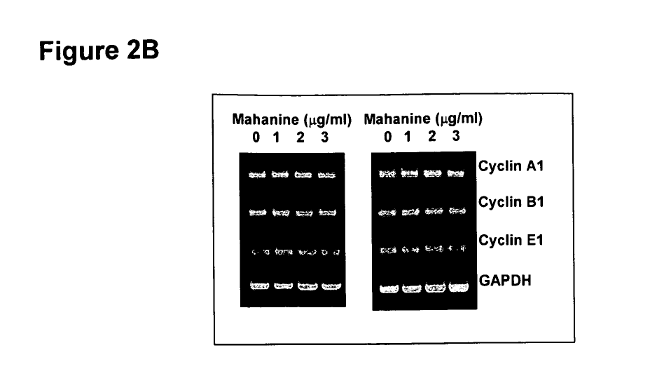

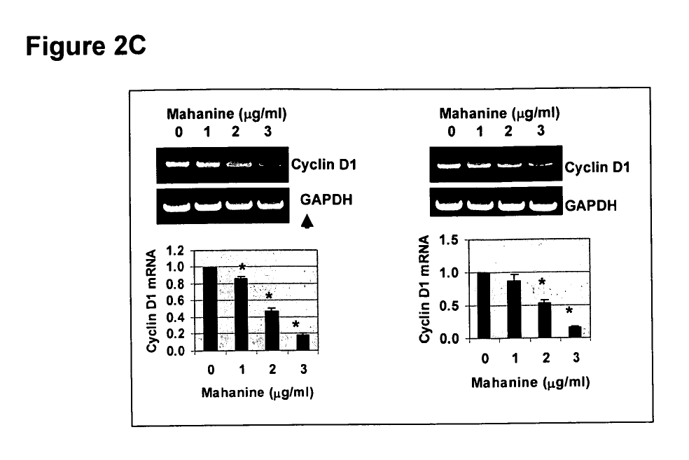

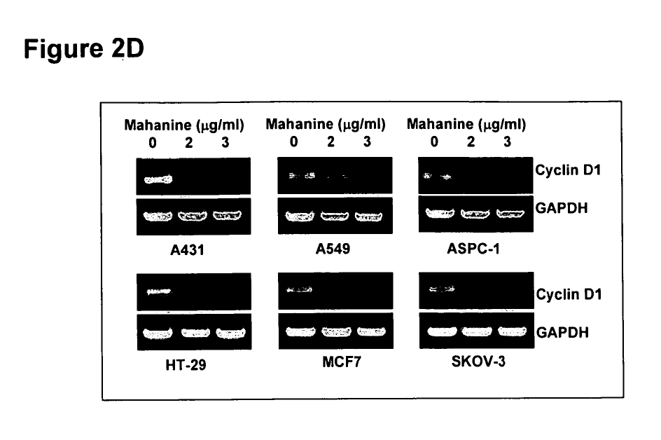

Assessment of the gene array data showed that mahanine down-regulated the expression of cyclin D1 (FIG. 2A). To confirm this, Applicants examined the expression levels of cyclin D1 by RT-PCR in PC-3 cells treated with various concentrations of mahanine for 3 days. Mahanine down-regulated the expression of cyclin D1 in PC3 cells in a dose-dependent manner (FIG. 2C). Similar results were also obtained with the LNCaP cells treated with various concentrations of mahanine (FIG. 2C). The expression of cyclin D1 was also assessed in various non-prostatic cancer cell lines. Results showed similar effects of mahanine treatment: down regulation of cyclin D1 in epidermoid, lung, pancreatic, colon, breast and ovarian cancer cells (FIG. 2D). In contrast to the expression of cyclin D1, mahanine treatment did not alter the expression of cyclin A1, B1 or E1 in PC3 or LNCaP cells (FIG. 2B). These results suggest that mahanine adversely affects the expression of cyclin D1 and not other cyclins.

### Example 3

### Mahanine Down-Regulates Cyclin D1 Protein Levels in Human Prostate Cancer Cells

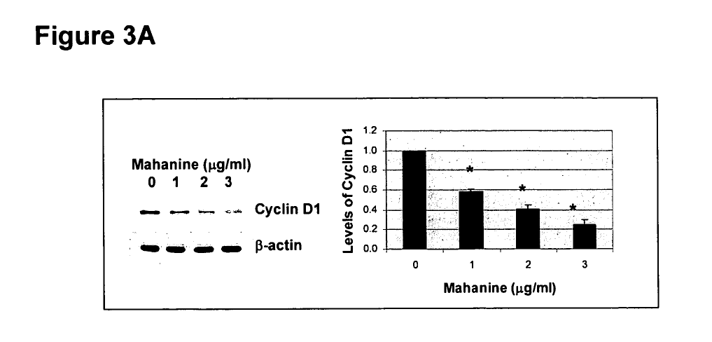

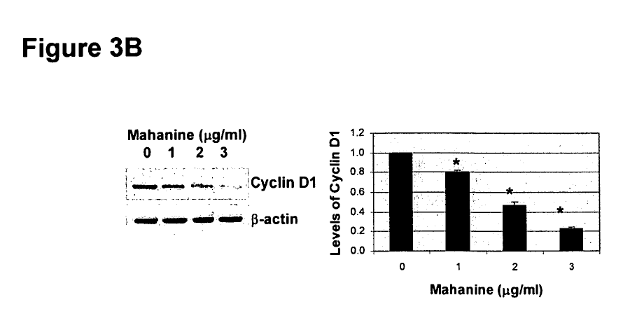

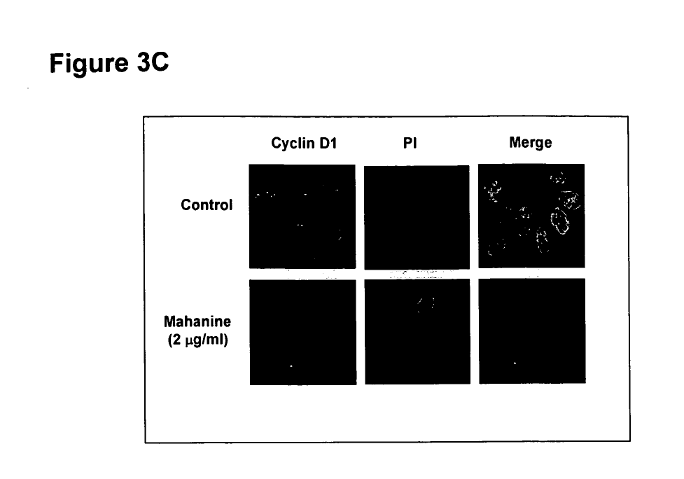

Since mahanine inhibits cyclin D1 expression in prostate a well as other non-prostatic cancer cell lines, Applicants were interested in examining the effect of mahanine on cyclin D1 protein levels in prostate cancer cells after mahanine treatments. As expected, Applicants observed a dose-dependent decrease in cyclin D1 protein in PC3 cells. Even a lower dose of mahanine (1 μg/ml) decreased cyclin D1 protein levels to about 40%, which was statistically significant (p<0.01) (FIG. 3A). Similar results were also obtained with LNCaP cells (FIG. 3B). Since the localization of cyclin D1 in the nucleus is necessary for the progression of the cell cycle, Applicants examined its localization in PC3 cells with or without mahanine (2 μg/ml) treatments for 2 days. In vehicle-treated control cells, cyclin D1 protein was present predominantly in the nucleus. Mahanine treatment reduced and/or prevented the nuclear localization of cyclin D1 (FIG. 3C). Negligible staining of cyclin D1 was observed in the cytoplasm. These results suggest that treatment of mahanine is associated with the decreased levels of cyclin D1 and prevents its nuclear localization.

### Example 4

### Mahanine Arrests Human Prostate Cancer Cells at G0/G1-Phase of Cell Cycle

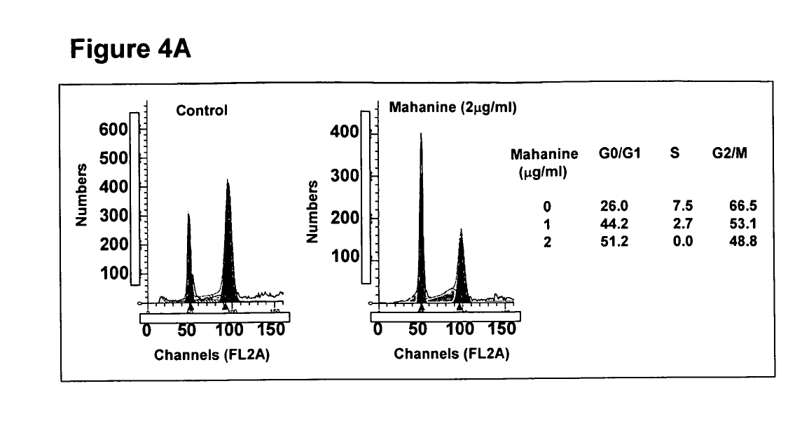

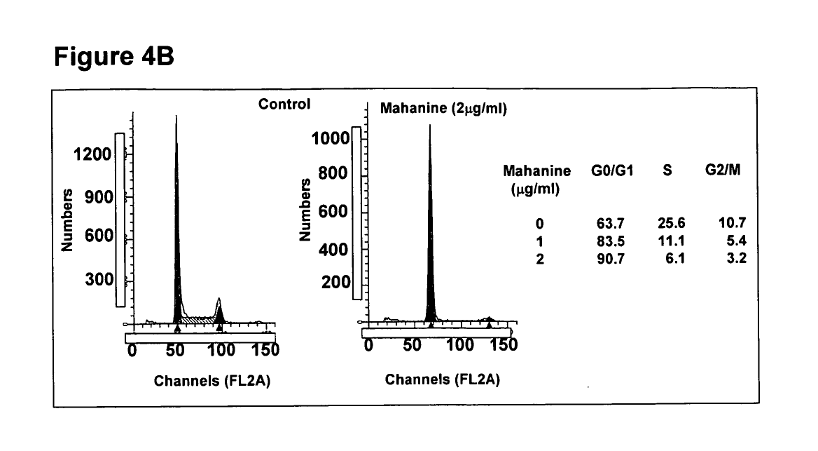

Since cyclin D1 regulates cell cycle progression at the G0/G1 phase, Aplicants examined the cell cycle profile of PC3 and LNCaP cells in the presence of various concentrations of mahanine. As seen in FIG. 4A, 26%, 7.5% and 66.5% cells were at G0/G1-, S- and G2/M-phases, respectively. One microgram mahanine treatment increased the G0/G1-phase and decreased the S-phase in PC3 cells. G0/G1-phase was further increased with 2 μg/ml mahanine treatment, while S-phase cells were completely abolished. Similar effects were observed in LNCaP cells (FIG. 4B). G0/G1 phase was increased from 63.7% to 90.7% and S-phase was decreased from 25.6% to 6.1%. These results suggest that mahanine reduces DNA synthesis and arrests prostate cancer cells at the G0/G1-phase of cell cycle.

### Example 5

### RASSF1A Down-Regulates Cyclin D1 Expression in Human Prostate Cancer Cells

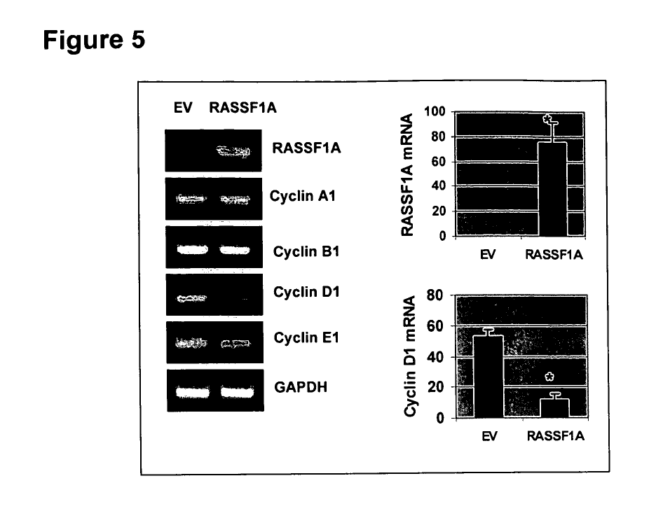

Mahanine induced RASSF1A and inhibited cyclin D1 transcription and, therefore, Applicants were interested in determining whether RASSF1A is involved in the regulation of cyclin D1 expression. After transient transfection of RASSF1A for 3 days, cyclin D1, A1, B1 and E1 were examined by RT-PCR. FIG. 5 shows that the expression of RASSF1A decreased cyclin D1 mRNA levels by approximately 5-fold, but did not affect cyclin A1, B1 or E1. This result suggests that RASSF1A specifically regulates cyclin D1 expression in prostate cancer cells.

### Example 6

### Mahanine Reduces Cyclin D1 Promoter Activity while RASSF1A siRNA Abolishes Cyclin D1 Promoter Activity in Human Prostate Cancer Cells

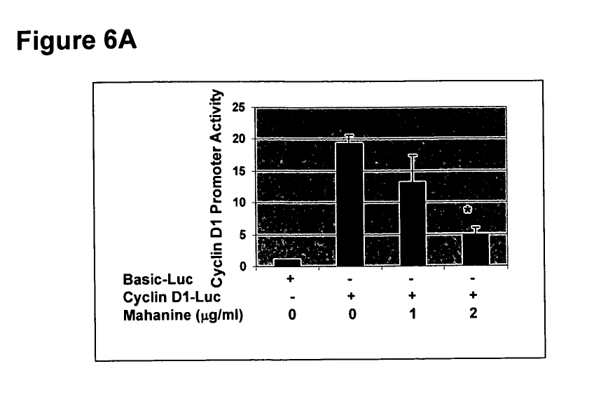

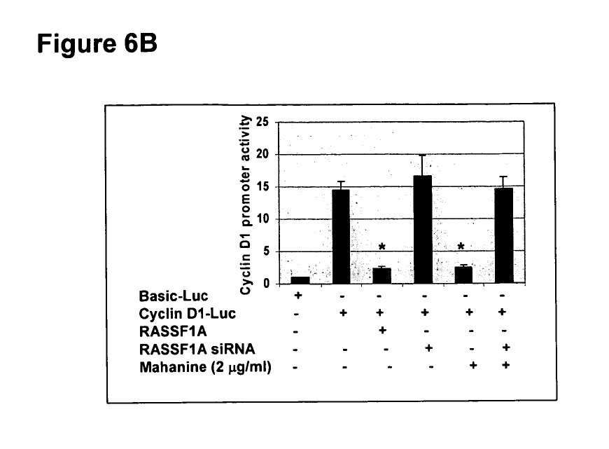

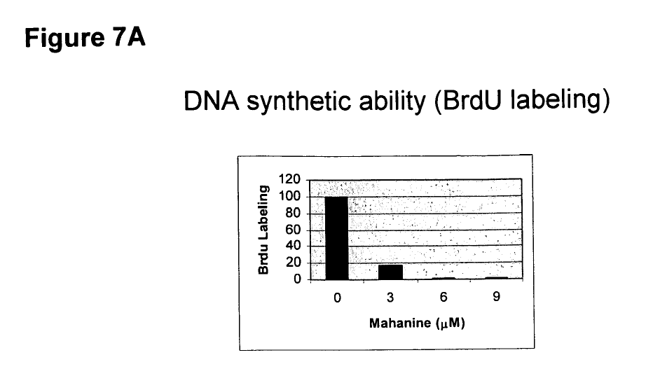

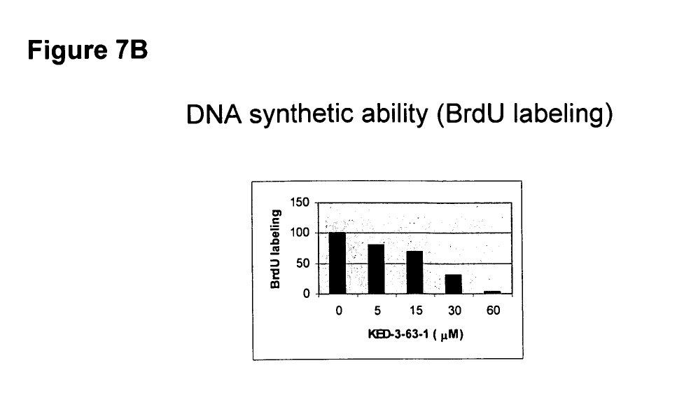

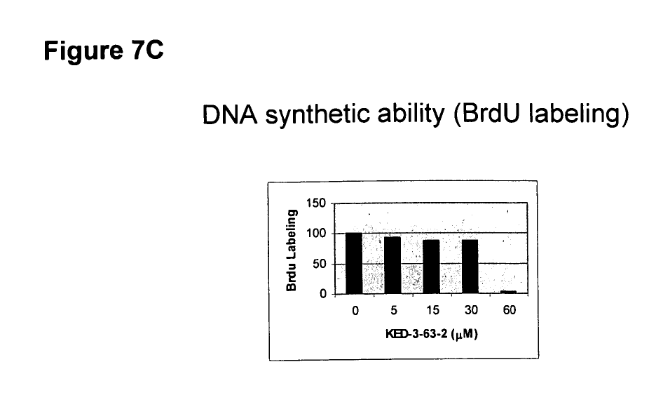

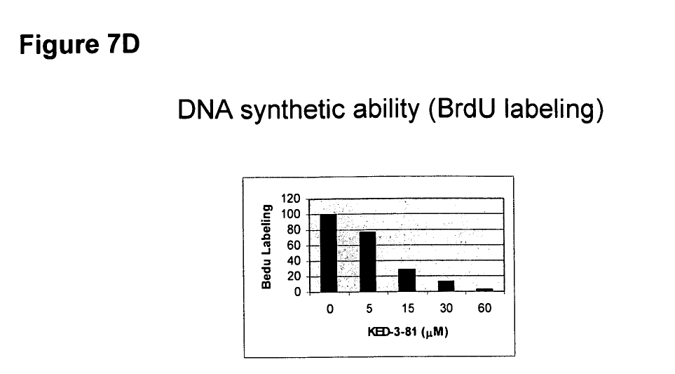

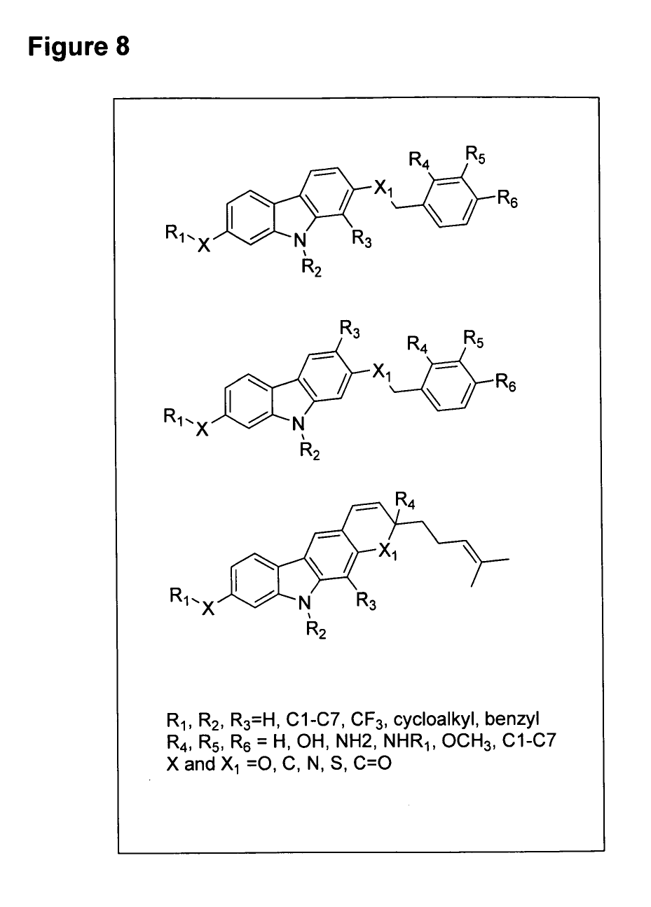

As described, Applicants demonstrated that mahanine inhibits cyclin D1 expression in prostate cancer cells and various other non-prostatic cancer cells. They also examined whether mahanine represses the transcriptional activity of cyclin D1. To this end, full-length cyclin D1 promoter luciferase construct was transiently transfected in PC3 cells for 48 hours and then treated for another 48 hours with various concentrations of mahanine. As seen in FIG. 6A, cyclin D1 promoter activity in mahanine-treated PC3 cells was approximately 20-fold that in basic vector transfected cells. Mahanine treatment decreased cyclin D1 promoter activity in a dose-dependent manner and to a significant extent (P<0.01), demonstrating the regulation of cyclin D1 transcriptional activity by mahanine.

A cyclin D1 promoter activity assay was used to determine the relationship between mahanine-induced RASSF1A expression and down-regulation of cyclin D1 transcriptional activity. As seen in FIG. 6B, similarly to mahanine treatment, transfection of RASSF1A decreased cyclin D1 promoter activity significantly (p<0.001). On the other hand, the transfection of RASSF1A siRNA alone did not cause any significant change in cyclin D1 promoter activity; transfection of RASSF1A siRNA prevented the mahanine-induced repression of cyclin D1 promoter activity. These results clearly suggest that mahanine induces RASSF1A in prostate cancer cells and RASSF1A in turn represses cyclin D1 expression.

### Example 7

### Synthesis of Carbazole Compounds

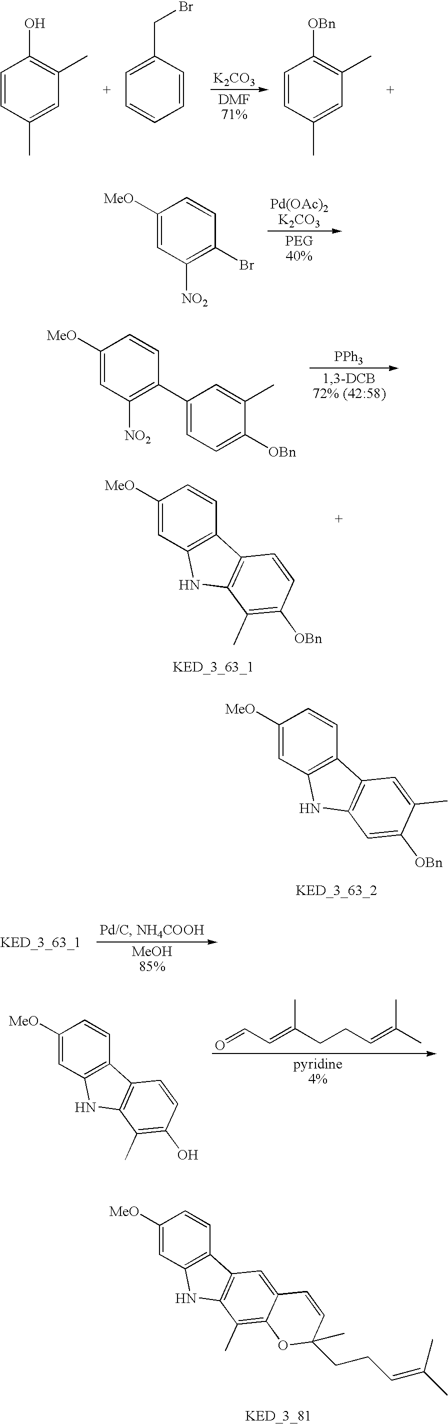

KED-3-63-1 and KED-3-63-2 were synthesized as follows: 4-iodo-2-methylphenol was benzyl-protected by treatment with benzyl bromide in the presence of K₂CO₃ in DMF to give 1-benzyloxy-4-iodo-2-methylbenzene in 71% yield. Palladium-mediated coupling of 1-benzyloxy-4-iodo-2-methyl benzene with 4-bromo-3-nitroanisole under basic conditions in PEG gave the homologated product in 40% yield. The carbazole was then formed by reduction of the nitro group and concomitant cycloaddition by treatment with PPh₃ in 1,3-dichlorobenzene. KED-3-63-1 and KED-3-63-2 were formed as a 42:58 mixture, respectively, in an overall 72% yield. The mixture was separated by column chromatography.

KED-3-81 was then synthesized from KED-3-63-1 in two steps. Debenzylation of KED-3-81 was effected by treatment with a methanolic solution of NH₄CO₂H in the presence of Pd—C. The resulting phenolic compound was treated with citral in pyridine at elevated temperatures to give KED-3-81 in 4% yield. Spectral data given below are consistent with the structures of each compound listed.

### NMR of Carbazole Compounds:

KED-3-63-1: ¹H NMR (400 MHz, CDCl₃): δ 7.84 (d, J=8.4 Hz, 1H), 7.76 (s, 1H), 7.73 (d, J=8.4 Hz, 1H), 7.50 (d, J=7.6 Hz, 2H), 7.41 (t, J=7.6 Hz, 2H), 7.34 (m, 1H), 6.93 (d, J=2.0 Hz, 1H), 6.91 (d, J=8.4 Hz, 1H), 6.83 (dd, J_{S}=2.0 Hz, J_{L}=8.4 Hz, 1H), 5.18 (s, 2H), 3.90 (s, 3H), 2.44 (s, 3H); ¹³C NMR (100.6 MHz, CDCl₃): δ 158.21, 154.40, 141.09, 140.27, 137.76, 128.46, 127.71, 127.29, 120.33, 117.90, 117.67, 116.88, 107.96, 107.69, 106.14, 95.00, 71.33, 55.60, 10.12; mp 167-168° C.

KED-3-63-2: ¹H NMR (400 MHz, CDCl₃): δ 7.80 (d, J=8.4 Hz, 1H), 7.76 (s, 1H), 7.72 (s, 1H), 7.49 (d, J=7.2 Hz, 2H), 7.41 (t, J=7.2 Hz, 2H), 7.33 (m, 1H), 6.89 (s, 1H), 6.87 (d, J=2.4 Hz, 1H), 6.81 (dd, J_{S}=2.4 Hz, J_{L}=8.4 Hz, 1H), 5.16 (s, 2H), 3.88 (s, 3H), 2.42 (s, 3H); ¹³C NMR (100.6 MHz, DMSO-d₆): δ 157.25, 154.53, 140.69, 139.13, 137.56, 128.33, 127.51, 127.11, 120.38, 119.64, 117.34, 116.30, 115.66, 107.05, 94.47, 94.26, 69.20, 55.10, 16.59; mp 250-251° C.

KED-3-81: ¹H NMR (400 MHz, CDCl₃): δ 7.77 (d, J=8.4 Hz, 1H), 7.74 (s, 1H), 7.40 (s, 1H), 6.90 (d, J=2.4 Hz, 1H), 6.80 (dd, J_{S}=2.4 Hz, J_{L}=8.4 Hz, 1H), 6.51 (d, J=10.0 Hz, 1H), 5.54 (d, J=10.0 Hz, 1H), 5.11 (m, 1H), 3.88 (s, 3H), 2.35 (s, 3H), 2.16 (m, 2H), 1.73 (m, 2H), 1.65 (s, 3H), 1.57 (s, 3H), 1.43 (s, 3H); ¹³C NMR (100.6 MHz, CDCl₃): δ 131.51, 129.02, 127.44, 124.32, 124.09, 120.06, 118.24, 116.31, 115.10, 114.44, 107.73, 104.77, 95.11, 78.31, 55.64, 41.04, 27.31, 26.13, 25.66, 22.76, 21.70, 17.57, 9.51.

### Example 8

### Biological Function of Novel Carbazole Compounds

Anticancer effects and activation of a tumor suppressor gene, RASSF1A) KED-3-63-1, KED 3-63-2 and KED 3-81 were assessed. Results showed that KED-3-63-1 and KED-3-81 have anti-cancer effects and that KED-3-63-2 does not. KED-3-63-1 induced RASSF1A. As a result, a key cell cycle regulator, cyclin D1 expression is down-regulated.

### REFERENCES

1. Dammann R, Li C, Yoon J H, Chin P L, Bates S, Pfeifer G P. Epigenetic inactivation of a RAS association domain family protein from the lung tumour suppressor locus 3p21.3. Nat Genet. 2000; 25:315-9.
2. Burbee D G, Forgacs E, Zochbauer-Muller S, Shivakumar L, Fong K, Gao B, Randle D, Kondo M, Virmani A, Bader S, Sekido Y, Latif F, Milchgrub S, Toyooka S, Gazdar A F, Lerman M I, Zabarovsky E, White M, Minna J D. Epigenetic inactivation of RASSF1A in lung and breast cancers and malignant phenotype suppression. J Natl Cancer Inst 2001; 93:691-9.
3. Vos M D, Ellis C A, Bell A, Birrer M J, Clark G J. Ras uses the novel tumor suppressor RASSF1 as an effector to mediate apoptosis. J Biol Chem 2000; 275:35669-72.
4. Pfeifer G P, Yoon J H, Liu L, Tommasi S, Wilczynski S P, Dammann R. Methylation of the RASSF1A gene in human cancers. Biol Chem 2002; 383:907-14. Review.
5. Dammann R, Schagdarsurengin U, Strunnikova M, Rastetter M, Seidel C, Liu L, Tommasi S, Pfeifer G P. Epigenetic inactivation of the Ras-association domain family 1 (RASSF1A) gene and its function in human carcinogenesis. Histol Histopathol 2003; 18:665-77.
6. Dammann R, Takahashi T, Pfeifer G P. The CpG island of the novel tumor suppressor gene RASSF1A is intensely methylated in primary small cell lung carcinomas. Oncogene 2001; 20:3563-7.
7. Dammann R, Yang G, Pfeifer G P. Hypermethylation of the cpG island of Ras association domain family 1A (RASSF1A), a putative tumor suppressor gene from the 3p21.3 locus, occurs in a large percentage of human breast cancers. Cancer Res 2001; 61:3105-9.
8. Yan P S, Shi H, Rahmatpanah F, Hsiau T H, Hsiau A H, Leu Y W, Liu J C, Huang T H. Differential distribution of DNA methylation within the RASSF1A CpG island in breast cancer. Cancer Res 2003; 63:6178-86.
9. Schagdarsurengin U, Wilkens L, Steinemann D, Flemming P, Kreipe H H, Pfeifer G P, Schlegelberger B, Dammann R. Frequent epigenetic inactivation of the RASSF1A gene in hepatocellular carcinoma. Oncogene 2003; 22:1866-71.
10. Zhang Y J, Ahsan H, Chen Y, Lunn R M, Wang L Y, Chen S Y, Lee P H, Chen O, Santella R M. High frequency of promoter hypermethylation of RASSF1A and p16 and its relationship to aflatoxin B1-DNA adduct levels in human hepatocellular carcinoma. Mol Carcinog 2002; 35:85-92.
11. Zhong S, Yeo W, Tang M W, Wong N, Lai P B, Johnson P J. Intensive hypermethylation of the CpG island of Ras association domain family 1A in hepatitis B virus-associated hepatocellular carcinomas. Clin Cancer Res 2003; 9:3376-82.
12. Liu L, Broaddus R R, Yao J C, Xie S, White J A, Wu T T, Hamilton S R, Rashid A. Epigenetic alterations in neuroendocrine tumors: methylation of RAS-association domain family 1, isoform A and p16 genes are associated with metastasis. Mod Pathol 2005; 18:1632-40.
13. Lo K W, Kwong J, Hui A B, Chan S Y, To K F, Chan A S, Chow L S, Teo P M, Johnson P J, Huang D P. High frequency of promoter hypermethylation of RASSF1A in nasopharyngeal carcinoma. Cancer Res 2001; 61:3877-81.
14. Dreijerink K, Braga E, Kuzmin I, Geil L, Duh F M, Angeloni D, Zbar B, Lerman M I, Stanbridge E J, Minna J D, Protopopov A, Li J, Kashuba V, Klein G, Zabarovsky E R. The candidate tumor suppressor gene, RASSF1A, from human chromosome 3p21.3 is involved in kidney tumorigenesis. Proc Natl Acad Sci USA 2001; 98:7504-9.
15. Lee M G, Kim H Y, Byun D S, Lee S J, Lee C H, Kim J I, Chang S G, Chi S G. Frequent epigenetic inactivation of RASSF1A in human bladder carcinoma. Cancer Res 2001; 61:6688-92.
16. Kuzmin I, Gillespie J W, Protopopov A, Geil L, Dreijerink K, Yang Y, Vocke C D, Duh F M, Zabarovsky E, Minna J D, Rhim J S, Emmert-Buck M R, Linehan W M, Lerman M I. The RASSF1A tumor suppressor gene is inactivated in prostate tumors and suppresses growth of prostate carcinoma cells. Cancer Res 2002; 62:3498-502.
17. Liu L, Yoon J H, Dammann R, Pfeifer G P. Frequent hypermethylation of the RASSF1A gene in prostate cancer. Oncogene 2002; 21:6835-40.
18. Maruyama R, Toyooka S, Toyooka K O, Virmani A K, Zochbauer-Muller S, Farinas A J, Minna J D, McConnell J, Frenkel E P, Gazdar A F. Aberrant promoter methylation profile of prostate cancers and its relationship to clinicopathological features. Clin Cancer Res 2002; 8:514-9.
19. Chow L S, Lo K W, Kwong J, To K F, Tsang K S, Lam C W, Dammann R, Huang D P. RASSF1A is a target tumor suppressor from 3p21.3 in nasopharyngeal carcinoma. Int J Cancer 2004; 109:839-47.
20. Li J, Wang F, Protopopov A, Malyukova A, Kashuba V, Minna J D, Lerman M I, Klein G, Zabarovsky E. Inactivation of RASSF1C during in vivo tumor growth identifies it as a tumorsuppressor gene. Oncogene 2004; 23:5941-9.
21. Hesson L, Bieche I, Krex D, Criniere E, Hoang-Xuan K, Maher E R, Latif F. Frequent epigenetic inactivation of RASSF1A and BLU genes located within the critical 3p21.3 region in gliomas. Oncogene 2004; 23:2408-19.
22. Khokhlatchev A, Rabizadeh S, Xavier R, Nedwidek M, Chen T, Zhang X F, Seed B, Avruch J. Identification of a novel Ras-regulated proapoptotic pathway. Curr Biol 2002; 12:253-65.
23. Liu L, Tommasi S, Lee D H, Dammann R, Pfeifer G P. Control of microtubule stability by the RASSF1A tumor suppressor. Oncogene 2003; 22:8125-36.
24. Song M S, Song S J, Ayad N G, Chang J S, Lee J H, Hong H K, Lee H, Choi N, Kim J, Kim H, Kim J W, Choi E J, Kirschner M W, Lim D S. The tumour suppressor RASSF1A regulates mitosis by inhibiting the APC-Cdc20 complex. Nat Cell Biol 2004; 6:129-37.
25. Jackson P K. Linking tumor suppression, DNA damage and the anaphase-promoting complex. Trends Cell Biol 2004; 14:331-4. Review.
26. Rong R, Jin W, Zhang J, Sheikh M S, Huang Y. Tumor suppressor RASSF1A is a microtubule-binding protein that stabilizes microtubules and induces G2/M arrest. Oncogene 2004; 23:8216-30.
27. Tommasi S, Dammann R, Zhang Z, Wang Y, Liu L, Tsark W M, Wilczynski S P, Li J, You M, Pfeifer G P. Tumor susceptibility of Rassf1a knockout mice. Cancer Res 2005; 65:92-8.
28. Shivakumar L, Minna J, Sakamaki T, Pestell R, White M A. The RASSF1A tumor suppressor blocks cell cycle progression and inhibits cyclin D1 accumulation. Mol Cell Biol 2002; 22:4309-18.
29. Ahmed-Choudhury J, Agathanggelou A, Fenton S L, Ricketts C, Clark G J, Maher E R, Latif F. Transcriptional regulation of cyclin A2 by RASSF1A through the enhanced binding of p120E4F to the cyclin A2 promoter. Cancer Res 2005; 65:2690-7.
30. Whang Y M, Kim Y H, Kim J S, Yoo Y D. RASSF1A suppresses the c-Jun-NH2-kinase pathway and inhibits cell cycle progression. Cancer Res 2005; 65:3682-90.
31. Donn A S, Muir C S. Prostatic cancer: some epidemiological features. Bull Cancer 1985; 72:381-290.
32. Wingo P A, Landis S, Ries L A. An adjustment to the 1997 estimate for new prostate cancer cases. CA Cancer J Clin 1997; 47:239-242.
33. Sinha S, Pal B C, Jagadeesh S, Banerjee P P, Bandyopadhaya, and Bhattacharya S. Mahanine inhibits growth and induces apoptosis in prostate cancer cells through the deactivation of Akt and activation of caspases. Prostate 2006; 661257-65.
34. Lee M G, Kim H Y, Byun D S, Lee S J, Lee C H, Kim J I, Chang S G, Chi S G. Frequent epigenetic inactivation of RASSF1A in human bladder carcinoma. Cancer Res 2001; 61:6688-92.
35. Albanese C, Johnson J, Watanabe G, Eklund N, Vu D, Arnold A, Pestell R G. Transforming p21ras mutants and c-Ets-2 activate the cyclin D1 promoter through distinguishable regions. J Biol Chem 1995; 270:23589-97.
36. Song M S, Song S J, Ayad N G, Chang J S, Lee J H, Hong H K, Lee H, Choi N, Kim J, Kim H, Kim J W, Choi E J, Kirschner M W, Lim D S. The tumour suppressor RASSF1A regulates mitosis by inhibiting the APC-Cdc20 complex Nat Cell Biol. 2004; 6:129-37.
37. M. Fu, C. Wang, Z. Li, T. Sakamaki, R. G. Pestell, Minireview: Cyclin D1: normal and abnormal functions. Endocrinology 2004; 145: 5439-47.
38. M. Shtutman, J. Zhurinsky, I. Simcha, C. Albanese, M. D'Amico, R. Pestell, A. Ben-Ze'ev, The cyclin D1 gene is a target of the beta-catenin/LEF-1 pathway. Proc Natl Acad Sci USA 1999; 96:5522-27.
39. J. A. Diehl, M. Cheng, M. F. Roussel, C. J. Sherr, Glycogen synthase kinase-3beta regulates cyclin D1 proteolysis and subcellular localization. Genes Dev 1998; 12: 3499-3511.
40. H. Koike, K. Suzuki, T. Satoh, N. Ohtake, T. Takei, S. Nakata, H. Yamanaka, Cyclin D1 gene polymorphism and familial prostate cancer: the AA genotype of A8700 polymorphism is associated with prostate cancer risk in men aged 70 years or older and metastatic stage. Anticancer Res 2003; 23: 4947-51.
41. M. Drobnjak, I. Osman, H. I. Scher, M. Fazzari, C. Cordon-Cardo, Overexpression of cyclin D1 is associated with metastatic prostate cancer to bone. Clin Cancer Res 2000; 6:1891-95.
42. Y. Chen, L. A. Martinez, M. LaCava, L. Coghlan, C. J. Conti, Increased cell growth and tumorigenicity in human prostate LNCaP cells by overexpression to cyclin D1. Oncogene 1998; 16:913-20.
43. T. C. Wang, R. D. Cardiff, L. Zukerberg, E. Lees, A. Arnold, E. V. Schmidt, Mammary hyperplasia and carcinoma in MMTV-cyclin D1 transgenic mice. Nature 1994; 369: 669-71.
44. C. J. Nelsen, D. G. Rickheim, N. A. Timchenko, M. W. Stanley, J. H. Albrecht. Transient expression of cyclin D1 is sufficient to promote hepatocyte replication and liver growth in vivo. Cancer Res 2001; 61:8564-68.
45. K. Nakayama, N. Ishida, M. Shirane, A. Inomata, T. Inoue, N. Shishido, I. Horii, D. Y. Loh, K. Nakayama, Mice lacking p27(Kip1) display increased body size, multiple organ hyperplasia, retinal dysplasia, and pituitary tumors. Cell 1996; 85:707-20.
46. T. Arato-Ohshima, H. Sawa, Over-expression of cyclin D1 induces glioma invasion by increasing matrix metalloproteinase activity and cell motility. Int J Cancer 1999: 83:387-92.
47. M. Drobnjak, I. Osman, H. I. Scher, M. Fazzari, C. Cordon-Cardo, Overexpression of cyclin D1 is associated with metastatic prostate cancer to bone. Clin Cancer Res 2000; 6:1891-95.
48. Khokhlatchev A, Rabizadeh S, Xavier R, Nedwidek M, Chen T, Zhang X F, Seed B, Avruch J. Identification of a novel Ras-regulated proapoptotic pathway. Curr Biol 2002; 12:253-65.
49. Lee K K, Ohyama T, Yajima N, Tsubuki S, Yonehara S. MST, a physiological caspase substrate, highly sensitizes apoptosis both upstream and downstream of caspase activation. J Biol Chem 2001; 276:19276-85.

1. A compound of the general formula: 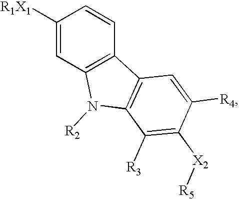
wherein:
R₁, R₂, R₃ and R₄ can be the same or different and each is H, alkyl, alkenyl, cycloalkyl, or benzyl, optionally substituted;
R₅ is alkyl, alkenyl, or an optionally substituted benzyl group;
or, R₄ and R₅ are joined together to form a six-membered ring, optionally substituted;
X₁ and X₂ can be the same or different and each is O, C(R₆)(R₇), NR₈, S, or C═O;
R₆ and R₇ can be the same or different and each is absent, H, alkyl, or alkenyl; and
R₈ is absent, H, alkyl, C═O, or S═O.

2. A compound according to claim 1, wherein X₁ and X₂ are O.

3. A compound according to claim 1, wherein R₂ is H.

4. A compound according to claim 1, wherein R₁ is CH₃ and R₅ is benzyl.

5. A compound according to claim 1, wherein R₃ is H or CH₃ and R₄ is H or CH₃.

6. A compound according to claim 1, wherein the compound comprises at least one benzyl group; the benzyl group comprising a phenyl ring optionally substituted with at least one OH, NH₂, NHR₉, OCH₃, or alkyl group, wherein R₉ is H, alkyl, C═O, or S═O.

7. A compound according to claim 1, wherein the compound is:

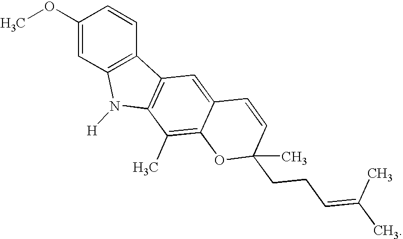

8. A compound according to claim 1, wherein the compound is:

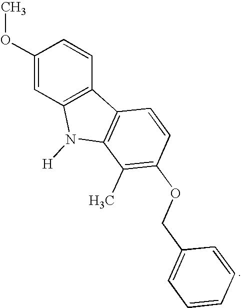

9. A compound according to claim 1, wherein the compound is:

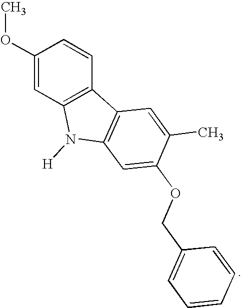

10. A method of inducing expression of an epigenetically silenced gene, RASSF1A, in cells, comprising contacting the cells with (a) mahanine, (b) a derivative equivalent or analogue of mahanine; (c) a compound of claim 1, 7, 8, or 9; or (d) a combination of two or more compounds of (a) (b) and/or (c).

11. The method of claim 10, wherein the cells are human cancer cells.

12. The method of claim 11, wherein the human cancer cells are prostate cancer cells, skin cancer cells, lung cancer cells, pancreatic cancer cells, colon cancer cells, breast cancer cells, or ovarian cancer cells.

13. The method of claim 12, wherein the cells are in an individual.

14. A method of treating an individual for cancer in which RASSF1A is epigenetically silenced, comprising administering to the individual a therapeutically effective amount of a drug that induces expression of RASSF1A in cancer cells or precancer cells in the individual, thereby limiting the extent to which a cancer in which RASSF1A is epigenetically silenced occurs in the individual or reversing (partially or completely) a cancer in which RASSF1A is epigenetically silenced in the individual.

15. The method of claim 14, wherein the cancer is prostate cancer, skin cancer, lung cancer, pancreatic cancer, colon cancer, breast cancer, ovarian cancer or other cancer in which RASSF1A is epigenetically silenced.

16. The method of claim 15, wherein the drug is (a) mahanine, (b) a derivative equivalent or analogue of mahanine; (c) a compound of claim 1, 7, 8, or 9; or (d) a combination of two or more compounds of (a) (b) and/or (c).

17. A method of treating prostate cancer in a man, comprising administering to the man a therapeutically effective amount of a drug (a) mahanine, (b) a derivative equivalent or analogue of mahanine; (c) a compound of claim 1, 7, 8, or 9; or (d) a combination of two or more compounds of (a) (b) and/or (c) whereby expression of epigenetically silenced RASSF1A is induced and prostate cancer occurs to a lesser extent than would be case in the absence of administration of mahanine of a derivative or analogue thereof.

18. The method of claim 17, wherein the drug is administered by a parenteral route, such as a subcutaneous, intramuscular, intraorbital, intracapsular, intraspinal, intrasternal, or intravenous route.

19. A method of identifying or screening for a drug that induces RASSF1A expression in cells in which RASSF1A is epigenetically silenced, comprising contacting the cells, referred to as test cells, with a candidate drug, under conditions appropriate for cell growth or maintenance and determining RASSF1A expression, wherein if RASSF1A expression is detected, the candidate drug is a drug that induces RASSF1A expression in the cells.

20. The method of claim 19, wherein the cells are cancer cells in which RASSF1A expression is epigenetically silenced.

21. The method of claim 20, wherein the test cells are prostate cancer cells, skin cancer cells, lung cancer cells, pancreatic cancer cells, colon cancer cells, breast cancer cells, or ovarian cancer cells.

22. The method of claim 21, further comprising determining RASSF1A expression in control cells wherein control cells are the same type of cancer cells as those contacted with the candidate drug and are maintained under the same conditions as the test cells except that they are not contacted with the candidate comparing RASSF1A expression in test cells with RASSF1A expression in control cells, wherein if RASSF1A expression in test cells is greater than RASSF1A expression in control cells, the candidate drug is a drug that induces RASSF1A expression in the test cells.

23. The method of claim 19 which is a method of identifying a drug that induces RASSF1A expression in prostate cancer cells and the candidate drug is a substituted carbazole derivative or a mahanine derivative or analogue.

24-30. (canceled)

31. The method of claim 10, wherein the compound is:

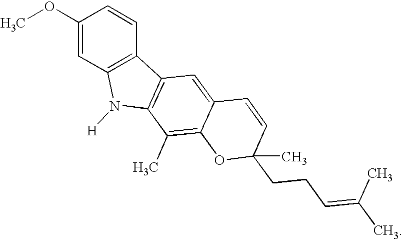

32. The method of claim 10, wherein the compound is:

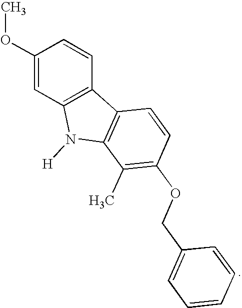

33. The method of claim 10, wherein the compound is:

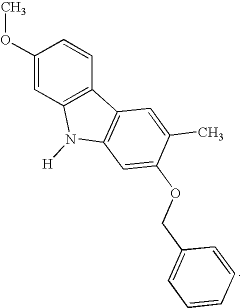

34. A pharmaceutical composition comprising a compound of claim 1 and an appropriate carrier.

35. A pharmaceutical composition comprising (a) a compound of claim 7, (b) a compound of claim 8, (c) a compound of claim 9 or a combination of two or more compounds of claim 7, claim 8 and/or claim 9.

36. The pharmaceutical composition of claim 34, additionally comprising at least one of the following: mahanine, a mahanine derivative and a mahanine equivalent.

37. A compound according to claim 2, wherein
R₂ is H;
R₃ is alkyl or alkenyl, optionally substituted;
R₄ is H, alkyl, or alkenyl, optionally substituted.

38. A compound according to claim 37, wherein R₁ is alkyl.

39. A compound according to claim 37, wherein R₁ is methyl.

40. A compound according to claim 37, wherein R₅ is alkyl, optionally substituted.

41. A compound according to claim 37, wherein R₅ is benzyl, optionally substituted.

42. A compound according to claim 37, wherein R₅ is benzyl.

43. A compound according to claim 37, wherein R4 and R5 are joined together to form a six-membered ring, optionally substituted.


SEQUENCE LISTING (10 sequences)

SEQ ID NO: 1 - DNA, 20 bp, artificial sequence
  synthetic polynucleotide, primer
cacacggactacaggggagt

SEQ ID NO: 2 - DNA, 20 bp, artificial sequence
  synthetic polynucleotide, primer
aggaagcggtccaggtagtt

SEQ ID NO: 3 - DNA, 20 bp, artificial sequence
  synthetic polynucleotide, primer
aaggagtgtgcgtcaggact

SEQ ID NO: 4 - DNA, 20 bp, artificial sequence
  synthetic polynucleotide, primer
caacgtgcagaagcctatga

SEQ ID NO: 5 - DNA, 20 bp, artificial sequence
  synthetic polynucleotide, primer
cgggaagtcactggaaacat

SEQ ID NO: 6 - DNA, 20 bp, artificial sequence
  synthetic polynucleotide, primer
ccgacccagaccaaagttta

SEQ ID NO: 7 - DNA, 20 bp, artificial sequence
  synthetic polynucleotide, primer
aagtggatggttccatttgc

SEQ ID NO: 8 - DNA, 20 bp, artificial sequence
  synthetic polynucleotide, primer
tttgatgccatccacagaaa

SEQ ID NO: 9 - DNA, 24 bp, artificial sequence
  synthetic polynucleotide, primer
ccacccatggcaaattccatggca

SEQ ID NO: 10 - DNA, 24 bp, artificial sequence
  synthetic polynucleotide, primer
tctagacggcaggtcaggtccacc